(19) United States
(12) Patent Application Publication
(10) Pub. No.: US 2012/0065506 A1
(43) Pub. Date: Mar. 15, 2012

(54) Mechanical, Electromechanical, and/or Elastographic Assessment for Renal Nerve Ablation

(76) Inventor: Scott Smith, Chaska, MN (US)

(21) Appl. No.: 13/228,233

(22) Filed: Sep. 8, 2011

Related U.S. Application Data
(60) Provisional application No. 61/381,796, filed on Sep. 10, 2010.

(51) Int. Cl. A61B 8/08

(52) U.S. Cl. 600/438; 600/439

(57) ABSTRACT

A transducer arrangement causes target tissue of the body to vibrate and senses resulting vibration of the target tissue. Changes in one or more mechanical properties of the target tissue are measured based on the sensed vibration. Changes in one or more electromechanical properties of the target tissue can also be measured based on the sensed vibration and various electrical parameters. An output indicative of the measured changes in the one or more mechanical and/or electromechanical properties of the target tissue is generated. Changes in elasticity of the target tissue, for example, can be measured based on the sensed vibration, such as changes resulting from ablation of the target tissue.

### SUMMARY

Embodiments of the disclosure are directed to methods and apparatuses for assessing one or more mechanical properties of tissue of the body. Embodiments of the disclosure are directed to methods and apparatuses for assessing one or more electromechanical properties of tissue of the body. Embodiments of the disclosure are directed to methods and apparatuses for assessing properties of tissue of the body using elastography or other technique in which stiffness or strain images of body tissue are acquired. Embodiments are directed to methods and apparatuses for assessing changes in one or more mechanical and/or electromechanical properties of tissue of the body due to ablation.

In accordance with various embodiments, methods and apparatuses of the disclosure involve causing target tissue of the body to vibrate and sensing vibration of the target tissue. Changes in one or more mechanical properties of the target tissue are measured based on the sensed vibration. Changes in one or more electromechanical properties of the target tissue can also be measured based on the sensed vibration and various electrical parameters. An output indicative of the measured changes in the one or more mechanical and/or electromechanical properties of the target tissue is generated. In some embodiments, changes in elasticity of the target tissue are measured based on the sensed vibration, such as changes resulting from ablation of the target tissue.

An apparatus, according to various embodiments, includes a catheter apparatus having a length sufficient to access target tissue of the body relative to a percutaneous access location. A transducer arrangement is supported at least in part by the catheter apparatus. The transducer arrangement includes a vibrating transducer configured to cause the target tissue to vibrate and a sensing transducer configured to sense vibration of the target tissue caused by the vibrating transducer. A detector is communicatively coupled to the transducer arrangement and configured to measure changes in one or more mechanical properties of the target tissue and produce an output signal indicative of the measured changes in the one or more mechanical properties. In various embodiments, the detector is configured to measure changes in elasticity of the target tissue and produce an output signal indicative of the measured changes in target tissue elasticity. The changes in the one or more mechanical properties, such as elasticity, may result from ablation of the target tissue.

According to other embodiments, an apparatus includes a catheter arrangement having a lumen and a length sufficient to access a patient's renal artery relative to a percutaneous access location. An ablation arrangement is configured to ablate perivascular renal nerve tissue. A transducer arrangement is supported at least in part by the catheter apparatus. The transducer arrangement includes a vibrating transducer configured to cause the perivascular renal nerve tissue to vibrate and a sensing transducer configured to sense vibration of the perivascular renal nerve tissue caused by the vibrating transducer. A detector is communicatively coupled to transducer arrangement and configured to measure changes in elasticity of the perivascular renal nerve tissue due to ablation and produce an output signal indicative of the measured changes in perivascular renal nerve tissue elasticity. The ablation arrangement may be integral to the catheter arrangement that supports the transducer arrangement or situated on a separate catheter.

Various embodiments of the disclosure are directed to methods and apparatuses that involve causing an electrode in contact with target tissue of the body to vibrate and sensing vibration of the electrode. Methods and apparatuses also involve measuring a force applied to the electrode caused by electrode vibration and measuring displacement of the electrode resulting from electrode vibration. An output indicative of the force applied to the electrode and the displacement of the electrode is produced, such as various values and waveforms.

An apparatus, according to further embodiments, includes a catheter apparatus having a length sufficient to access target tissue of the body relative to a percutaneous access location. An RF electrode is supported by the catheter apparatus and configured to contact the target tissue. A transducer arrangement is supported at least in part by the catheter apparatus. The transducer arrangement includes a vibrating transducer configured to emit acoustic energy that causes the RF electrode to vibrate and a sensing transducer configured to sense an acoustic wave indicative of displacement of the RF electrode caused by the emitted acoustic energy. A detector is communicatively coupled to the transducer arrangement. The detector is configured to generate an output indicative of a force applied to the RF electrode by the emitted acoustic energy and displacement of the RF electrode.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 illustrates a medical system including a medical device positioned within an organ, body of tissue, or cavity of the patient's body near the renal artery accessed via a natural orifice in accordance with various embodiments;

FIGS. 5 and 6 illustrate components of a tissue assessment arrangement that operate cooperatively to assess changes in a property of target tissue, such as tissue elasticity, during ablation in accordance with various embodiments;

FIG. 7 shows a system for assessing one or more mechanical properties of tissue of a vessel wall during ablation of tissue external to the vessel in accordance with various embodiments;

FIG. 8 illustrates the distal end of a catheter that incorporates a multiplicity of circumferentially spaced tissue assessment arrangements in accordance with various embodiments;

FIGS. 9 and 10 graphically show displacement and force (or applied electrical current) relationships for poor and good electrode-to-tissue contact scenarios in accordance with various embodiments;

FIGS. 11 and 12 graphically illustrate various waveforms being affected by good or poor RF electrode-to-tissue contact or by mechanical or conductivity changes in target tissue in accordance with various embodiments;

FIGS. 13-15 show changes in a mechanical vibration displacement waveform corresponding to changes in mechanical properties of target tissue during ablation, which is used to modulate an RF current waveform or electromechanical impedance waveform in accordance with various embodiments; and

FIG. 16 shows a representative renal ablation apparatus in accordance with various embodiments of the disclosure.

### DESCRIPTION

Embodiments of the disclosure are directed to apparatuses and methods for assessing one or more mechanical properties of tissue and changes in such properties due to ablation of target tissue of the body. Embodiments of the disclosure are directed to apparatuses and methods for assessing one or more mechanical properties of perivascular renal nerve tissue and changes in such properties due to ablation of the perivascular renal nerve tissue, such as for the treatment of hypertension. Embodiments of the disclosure are also directed to apparatuses and methods for assessing and monitoring RF electrode contact integrity with target tissue of the body, such as during RF ablation of the target tissue. Embodiments of the disclosure are directed to apparatuses and methods for assessing and monitoring RF electrode contact integrity with a wall of a patient's renal artery, such as during RF ablation of perivascular renal nerve tissue.

Ablation of perivascular renal nerves has been used for treatment of hypertension. Radiofrequency (RF) catheters positioned in the renal artery can be used to ablate perivascular renal nerves, but can cause damage to the renal artery. Control of ablation is important to effectively ablate the renal nerves while minimizing injury to the renal artery. Conventional RF ablation approaches simply apply energy for a predetermined time, and may monitor impedance or current, or temperature in the artery, but these parameters are typically suboptimal indicators of the effect of ablation on the target tissue. The effects of variable electrode contact with the artery wall, varying tissue properties, and variable anatomy, for example, can introduce unpredictability in the ablation effect on the target tissue.

Various embodiments take advantage of changes in tissue stiffness which occur as a result of ablation to monitor the progress of the ablation procedure. Monitoring tissue stiffness changes during ablation provides for accurate assessment of ablation effectiveness and avoidance of excess injury to non-target tissue. A variety of methodologies can be used for tissue stiffness assessment including, for example, 1-D elastography or M-Mode imaging, 2-D elastography, acoustic radiation force impulse imaging (ARFI), mechanical force and displacement assessment of a vibrating transducer, and changes in electromechanical impedance to assess electrode-to-tissue contact, among others.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

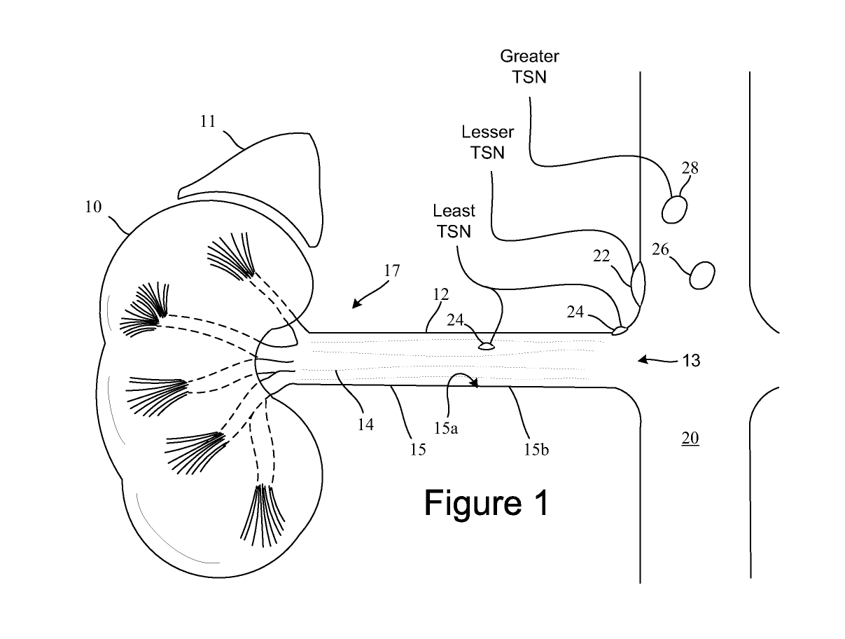

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

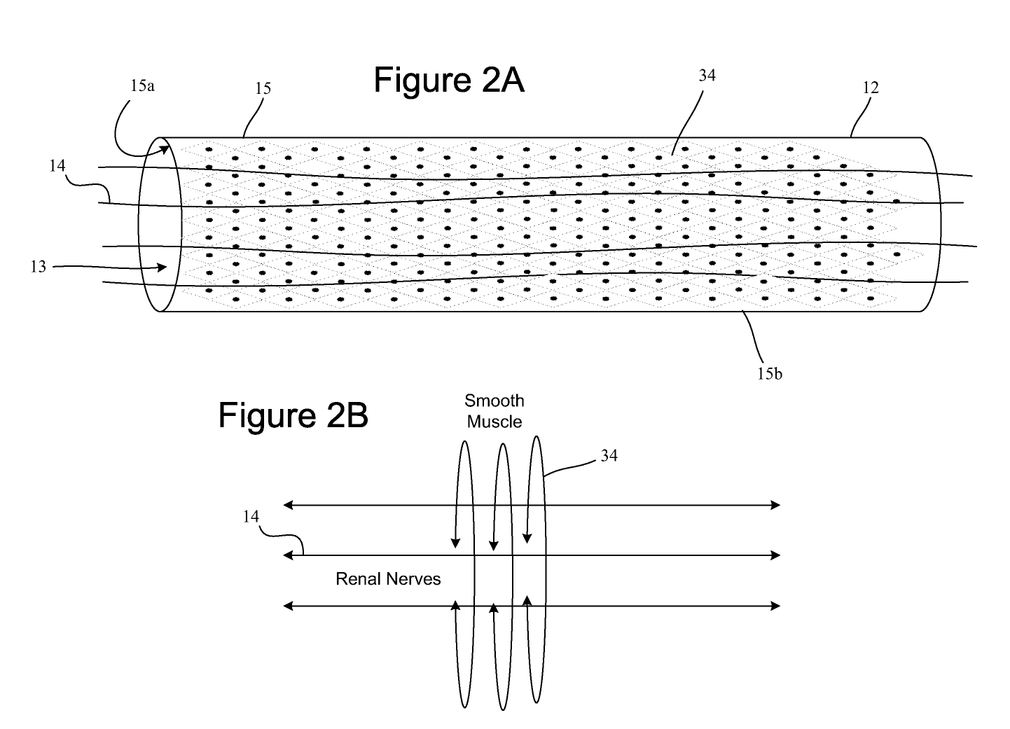

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

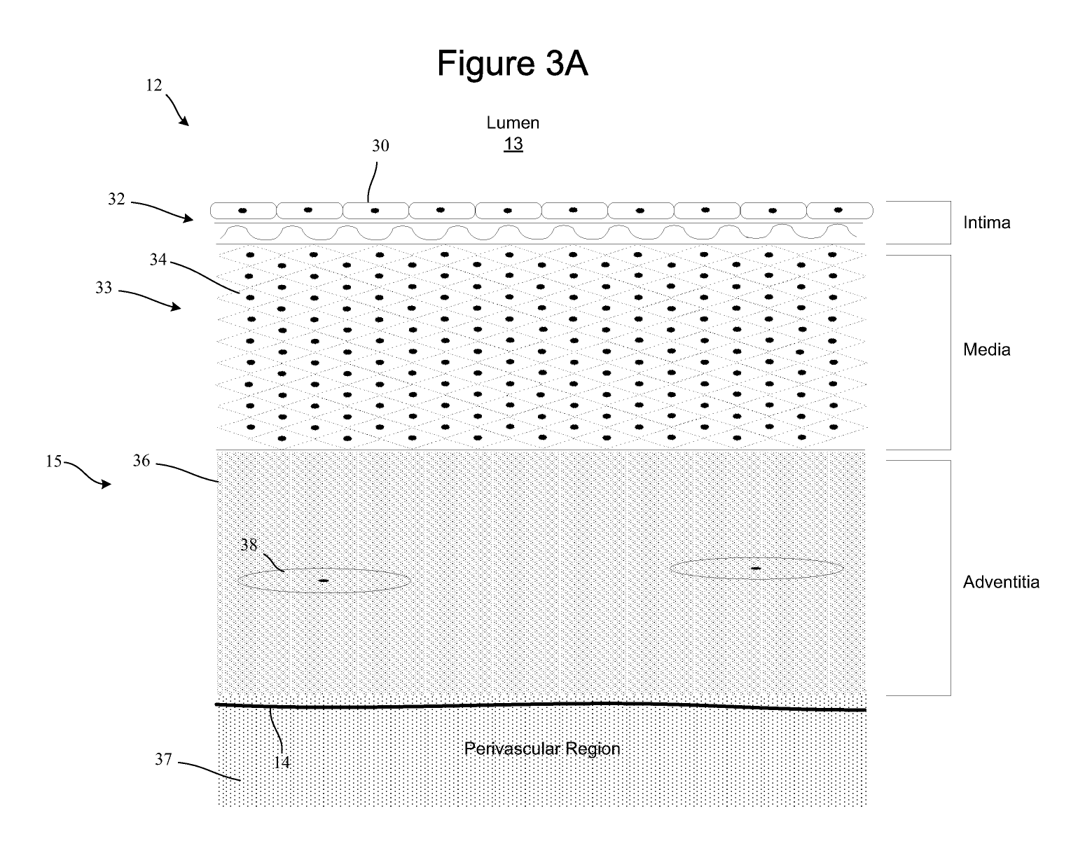

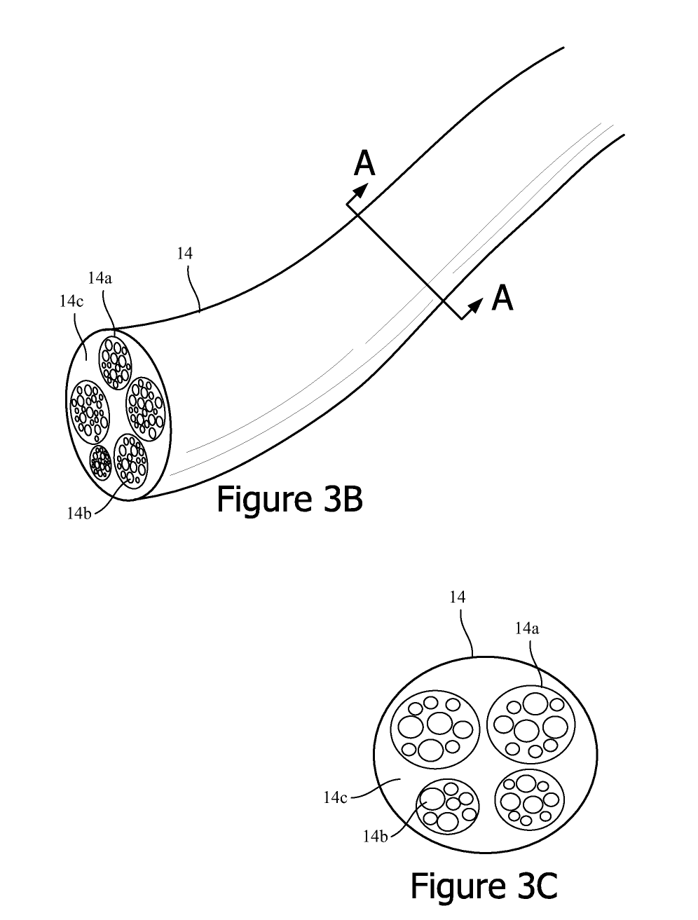

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

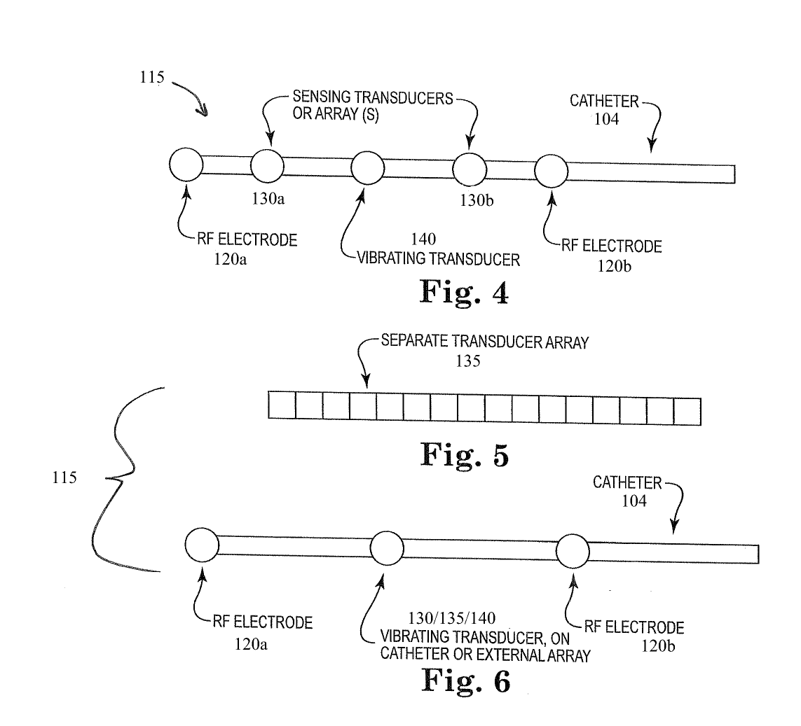

In accordance with various embodiments, and with reference to FIG. 4, a catheter 104 is configured to access a desired location of the body, such as a patient's renal artery 12. A tissue assessment arrangement 115 is provided at a distal end of the catheter 104. The tissue assessment arrangement 115 includes a number of components that operate cooperatively to assess changes in a property of target tissue that changes during and ablation procedure. The property of the target tissue monitored by the tissue assessment arrangement 115 is preferably one that changes during ablation and is reflective of the extent of ablation therapy delivered to the target tissue. A representative property of the target tissue that can be monitored to assess changes in target tissue during ablation is tissue elasticity. It is understood that other properties of the target tissue may be monitored to evaluate changes in the target tissue during ablation, and that more than one properties of the target tissue may be monitored and assessed in accordance with various embodiments.

The embodiment of the tissue assessment arrangement 115 shown in FIG. 4 includes a distal electrode 120a and a proximal electrode 120b which are configured to deliver high-frequency AC current to target tissue of the body. The distal and proximal electrodes 120a and 120b can be implemented as RF electrodes, for example. The distal and proximal electrodes 120a and 120b are preferably operated in a bipolar mode, although only one of the electrodes 120 need be included if operating in a unipolar mode. It is noted that a patient-external return electrode is used together with a single electrode 120 or tied electrodes when operating in a unipolar mode.

The tissue assessment arrangement 115 further includes a distal sensing transducer 130a and a proximal sensing transducer 130b. In the representative embodiment shown in FIG. 4, the pair of sensing transducers 130a and 130b are situated on the distal end of the catheter 104 between the pair of RF electrodes 120a and 120b. Although it is desirable to include a pair of sensing transducers 130a and 130a, it is understood that a single sensing transducer 130, such as a sensing transducer array, can be used. The tissue assessment arrangement 115 further includes a vibrating transducer 140 which, according to the embodiment of FIG. 4, is positioned at the distal end of the catheter 104 between the pair of sensing transducers 130a and 130b. It is understood that the relative positioning of the electrodes, sensing transducers, and vibrating transducer may differ from that shown in FIG. 4. It is noted that the terms “sensing transducer” and “vibrating transducer” in the context of embodiments of the disclosure refer to arrangements that may include one or a multiplicity of transducers.

In some embodiments, one or more vibration transducers 140 and/or sensing transducers 130 can be positioned at a different location in the body or external to the body. The vibrating and sensing transducers 140, 130 can be situated on the same catheter 104 or on separate catheters, for example. In other embodiments, a transducer array can be used to provide both vibrating and sensing functions. Various coatings or passive transponders can be used to aid in catheter localization when using an external transducer array.

According to various embodiments, one or more vibration transducers 140 are configured to direct high-frequency acoustic energy (e.g., >1 MHz, such as 1-100 MHz) to impinge target tissue, causing a change in a mechanical property of the target tissue which is manifested as a low-frequency return signal (e.g., <1K Hz, such as <100 Hz or 1-100 Hz) that is sensed by one or more sensing transducers 130. In other embodiments, one or more vibration transducers 140 are configured to direct low-frequency acoustic energy to impinge target tissue, causing a change in a mechanical property of the target tissue which can be sensed using one or more sensing transducers 130 operating at high-frequency. Various combinations of low and/or high-frequency source and return/imaging transducers are contemplated (e.g., low-frequency source and low-frequency return/imaging transducers; high-frequency source and high-frequency return/imaging transducers; high-frequency source and low-frequency return/imaging transducers; low-frequency source and high-frequency return/imaging transducers).

FIGS. 5 and 6 illustrate components of a tissue assessment arrangement 115 that operate cooperatively to assess changes in a property of target tissue, such as tissue elasticity, during ablation in accordance with various embodiments. The tissue assessment arrangement 115 according to various embodiments includes two separate components. A first component includes a vibrating transducer and a second component includes a separate sensing transducer arrangement. In some embodiments, one of the vibrating transducer and the sensing transducer arrangement is supported by a first catheter, and the other of the vibrating transducer and sensing transducer arrangement is supported by a second catheter. In other embodiments, one of the vibrating transducer and the sensing transducer arrangement is supported by a first catheter, and the other of the vibrating transducer and sensing transducer arrangement is configured as an external component.

For example, and according to one embodiment, at least one of the vibrating and sensing transducers 140, 130 is configured for extravascular or patient-external deployment. According to another embodiment, each of the vibrating and sensing transducers 140, 130 is configured for intravascular deployment. For example, one of the vibrating and sensing transducers 140, 130 can be deployed in a patient's renal artery, and the other of the vibrating and sensing transducers 140, 130 can be deployed in the body, such as the renal artery, a renal vein, or hepatic portal vein, for example. In some embodiments, one or both of the vibrating and sensing transducers 140, 130 can be deployed via renal nerve access achieved using a trans-hepatic route via the inferior vena cava and hepatic vein, similar to a TIPS procedure. In various embodiments, renal nerve access can be achieved using a body pathway that includes the inferior vena cava, hepatic vein, liver, and intraperitoneum.

According to some embodiments, and with further reference to FIGS. 5 and 6, a vibrating transducer 140 is situated at a distal end of a catheter 104, such as between a pair of electrodes 120a and 120b as shown in FIG. 6. As discussed previously, although a single vibrating transducer 140 is shown in the embodiment of FIGS. 5 and 6, two or more vibrating transducers 140 may be used. A sensing transducer, such as a transducer array 135, is situated on a second catheter or is implemented as an external component of the tissue assessment arrangement 115. In other embodiments, the transducer array 135 is configured as a vibrating transducer 140, and the transducer shown in FIG. 6 is configured as a sensing transducer 130. In some embodiments, the sensing transducer 130 may be implemented as a transducer array 135, and the vibrating transducer 140 may also be implemented as a transducer array 135.

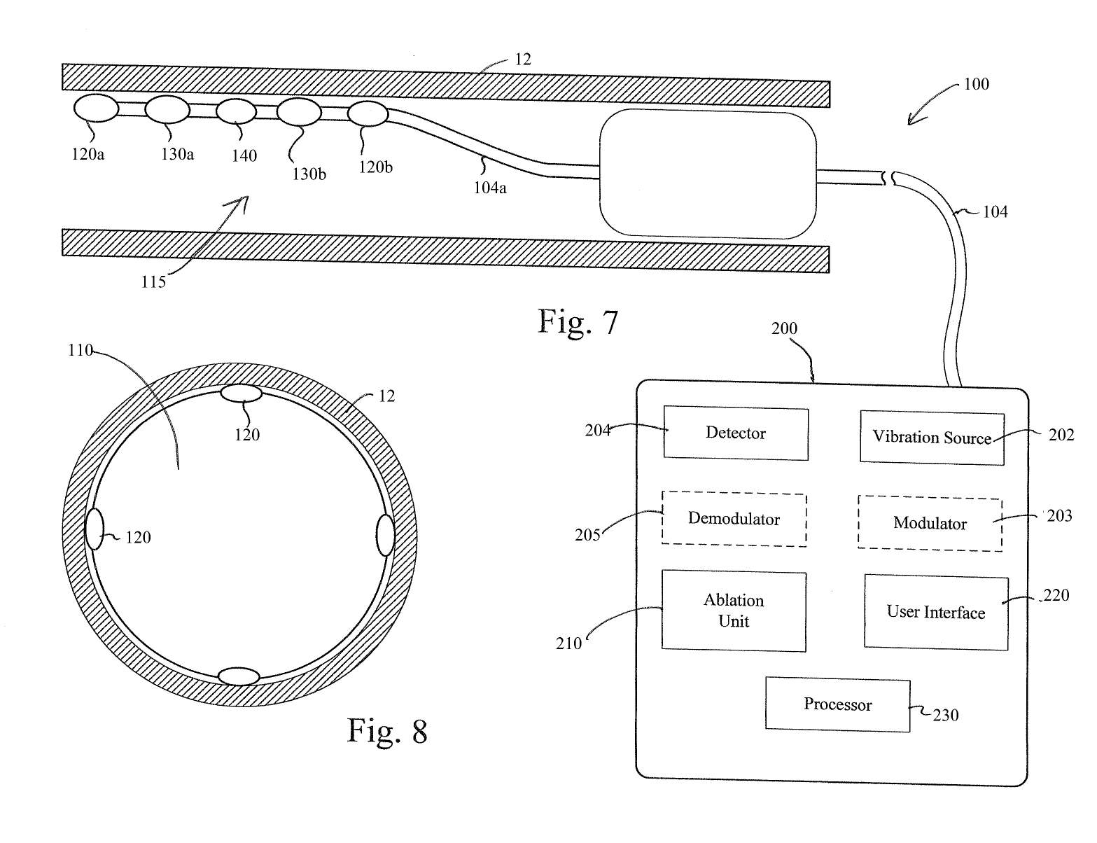

In accordance with embodiments configured for delivering ablation therapy to perivascular renal nerves, a catheter apparatus is configured for percutaneous access and navigation through a patient's arterial or venous systems. For example, the catheter 104 may have a length sufficient to access a patient's renal artery via the superior or inferior aorta from a percutaneous access location. A transducer arrangement 115 is supported at least in part by the catheter apparatus. The transducer arrangement includes a source transducer 140 configured to cause target tissue (e.g., perivascular renal nerves) to vibrate, and a sensing transducer arrangement 130 is configured to sense vibration of the target tissue caused by the source transducer 140. The sensed target tissue vibration information is communicated to a proximal end of the catheter apparatus and received by a detector (shown in FIG. 7) communicatively coupled to the transducer arrangement 115. The detector is configured to sense changes in tissue elasticity and produce an output signal indicative of the sensed changes in tissue elasticity. The detector can be controlled to measure changes in tissue elasticity continuously or intermittently during ablation of the target tissue.

During ablation, properties of the target tissue change, and this change can be detected using a tissue assessment arrangement 115 describe herein to monitor and assess the efficacy of the ablation procedure. In response to vibratory excitation of target tissue by a source signal generated by the vibrating transducer 140, the target tissue emits a return signal that is detected by the sensing transducer(s) 130. In some embodiments, changes in a mechanical property of the target tissue are sensed using scanning or imaging techniques. As ablation continues, the return signal emitted by the target tissue or the scanning/imaging data indicative of a mechanical property change in the tissue also changes. Changes in the return signal or scanning/imaging data are detected or acquired by the sensing transducers 130 and assessed by a detector communicatively coupled to the tissue assessment arrangement 115.

Changes in the target tissue and corresponding changes in the return signal or scanning/imaging data during an ablation procedure occur when the protein configuration of the target tissue changes due to ablation. In response to changes in the protein configuration of the target tissue, the tissue transmits, attenuates, absorbs, scatters, and/or reflects the source vibration signal differently. The pattern of vibration intensity in the target tissue can be monitored by one or more sensing transducers 130 on the catheter 104. In some embodiments, low-frequency mechanical vibrations, sonic, or ultrasonic vibrations, for example, can be used with an appropriate configuration of the transducers 130 and 140, vibration intensities, and timings. Low-frequency deformations induced in the target tissue can be used with high-frequency monitoring or imaging in accordance with various embodiments (e.g., 1-D or 2-D elastography). In some embodiments, for example, M-mode imaging can be used, in which ultrasound pulses are emitted in quick succession, and either an A-mode or B-mode image is taken for each succession.

According to various embodiments, the vibrating transducer 140 generates low-frequency acoustic waves that mechanically excite the target tissue. The vibrating transducer 140 may incorporate one or more mechanical excitation sources. A non-exhaustive list of representative mechanical excitation sources includes: a patient-external transducer (e.g., ˜10 Hz); catheter tip deflection (e.g., ˜10 Hz); balloon inflation oscillation; inertial elements in tip (e.g., axial, rotational); electroactive polymers (e.g., EAP); shape memory actuators; piezoelectric actuators; voice coils; and catheter shaft actuators (e.g., axial, rotational). In some embodiments, the vibrating transducer 140 need not be a component of the system, but rather a source within the body. For example, a patient's pulse pressure (e.g., ˜1 Hz) can provide for mechanical excitation of certain target tissue. Embodiments that exploit mechanical excitation sources of the body need not include a vibrating transducer 140.

As discussed previously, low-frequency deformations induced in the target tissue can be used with high-frequency monitoring or imaging in accordance with various embodiments, such as 1-D or 2-D elastography. Elastographic assessment of tissue provides for assessing changes in mechanical properties of tissue associated with ablation using low frequency deformation of tissue and imaging to quantify tissue deformation. According to embodiments that employ 1-D imaging (M-Mode) imaging, low-frequency deformation at about 60 Hz can be induced in the target tissue. Imaging can be performed using an intravascular ultrasound (IVUS) imaging system operating at a frequency of 7,680 Hz, for example. In this case, pulse echo ultrasound imaging is acquired repeatedly along the same vector through the tissue (A-line). The 2-D array of data formed by these sequential acquisitions may be analyzed using 2-D fast Fourier transforms (FFT) and/or cross correlation algorithms.

For data sampled at 100 MS/s and assuming a speed of sound of 1.5 mm/microsecond, for example, 1024 samples are sufficient to image to a depth of about 7.5 mm. In this illustrative example, 128 vectors will precisely image 1 complete deformation cycle. Thus, the complete 2-D array is 128×1024. It is desirable for efficient computer processing that the array size be an integral power of 2. Signal to noise ratio may be improved by extending the number of vectors to include multiple deformation cycles. For example, 8 cycles would result in a 1024×1024 array.

Two-dimensional or 2-D elastography is accomplished by extending the method described above using B-mode imaging. A subset of the R-Theta B-mode data would be resampled into a Cartesian matrix using any of a number of conventional interpolation algorithms.

Suitable IVUS imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; 6,945,938; and 6,254,541; as well as U.S. Patent Application Publication Nos. 20060253028; 20070016054; 20070038111; 20060173350; 20060100522; 20100179434; 2010002288; 20100249604; 20110071401; and 20110160586; all of which are incorporated herein by reference. Various other imaging approaches can be employed, such as optoacoustic imaging, optical coherence tomography, and angioscopy.

In accordance with various embodiments, the vibrating transducer 140 may be configured as a high-intensity focused ultrasound (e.g., HIFU) transducer. The sensing transducers 130 may be configured to sense low-frequency acoustic waves associated with low-frequency vibration of target tissue impinged by the higher frequency acoustic waves generated by the vibrating transducer 140.

According to various embodiments, a tissue assessment arrangement 115 can be implemented to include an acoustic radiation force transducer. This transducer exploits a physical phenomenon resulting from the interaction of an acoustic wave with an obstacle situated along its path. An acoustic radiation force transducer according to various embodiments can be implemented to measure the force exerted on target tissue by integrating the acoustic radiation pressure due to the presence of the acoustic wave over its time-varying surface.

When acoustic energy generated by the vibration transducer 140 impinges soft target tissue (e.g., perivascular renal nerve tissue), the acoustic energy is attenuated by the target tissue, largely by absorbing the acoustic energy. Because the soft target tissue cannot respond fast enough to positive and negative pressures transitions associated with the frequency of the acoustic energy, the motion of the soft target tissue becomes out of phase with the acoustic wave. As a result, energy is deposited into the target tissue causing a transfer of momentum in the direction of acoustic wave propagation. This momentum transfer generates a force that causes displacement of the soft target tissue. The timing associated with this displacement is significantly slower than that of the propagating acoustic wave generated by the vibration transducer 140.

Various aspects of the acoustic wave generated by the acoustic radiation force transducer 140, such as the magnitude, location, spatial extent, and duration of acoustic radiation force, can be controlled to interrogate the mechanical properties of the soft target tissue. For example, the target tissue can be excited at specific frequencies using the acoustic radiation force transducer 142 to evaluate the elasticity (e.g., viscoelasticity) properties of the soft target tissue.

In some embodiments, an acoustic radiation force impulse imaging (ARFI) technique can be used to assess tissue strain of the target tissue, by utilizing sound waves to interrogate the mechanical stiffness properties of target tissue. The frequency of an ARFI source typically ranges between about 10 MHz and 100 MHz, with 50 MHz representing a desirable operating frequency, with 1 μs pulses, for example. An ARFI technique can be used, for example, to assess changes in the mechanical stiffness or strain of renal artery wall tissue during ablation. As a lesion is being formed during ablation of target tissue such as the renal artery, for example, a catheter 104 implemented in accordance with embodiments of the disclosure can qualitatively visualize the target tissue for its stiffness relative to surrounding tissue, as well as the relative stiffness of its internal structure. Numerical measurements of the lesion can also be made using data received from the catheter apparatus 104 to provide a quantified assessment of the stiffness (and changes thereto) of the target tissue before, during, and after ablation.

A variety of acoustic radiation force transducer technologies can be adapted for use in assessing changes in tissue stiffness and/or other mechanical properties before, during, and after ablation. In addition to an acoustic radiation force impulse implementation discussed above, other useful implementations can include shearwave dispersion ultrasound vibrometry, spatially-modulated ultrasound radiation force (SMURF), supersonic shear imaging (SSI), and harmonic motion imaging (HMI), among others. Transducer arrangements that employ or facilitate use of these technologies can be implemented for use with various catheter apparatus embodiments described herein.

Turning now to FIG. 7, there is shown a system 100 for assessing one or more mechanical properties of vessel wall tissue during ablation of tissue external to the vessel in accordance with various embodiments. In the representative embodiment shown in FIG. 7, system 100 is configured for assessing one or mechanical properties of a renal artery 12 and/or perivascular renal nerve tissue adjacent the renal artery 12. The system 100 includes a catheter 104 which includes a tissue assessment arrangement 115 provided at its distal end. The configuration of the tissue assessment arrangement 115 shown in FIG. 7 is similar to that depicted in FIG. 4, and includes a pair of RF electrodes 130a and 130b, a pair of sensing transducers 120a and 120b, and a vibrating transducer 140.

Also provided at the distal end of the catheter 104 is a stabilizing mechanism 110, such as a balloon or expandable/collapsible mechanism suitable for deployment within the lumen of the renal artery 12. The section 104a of the catheter 104 distal of the stabilizing mechanism 110 is configured to establish contact with an inner surface of the renal artery 12 when the stabilizing mechanism 110 is in its deployed configuration (as is shown in FIG. 7). The distal end 104a of the catheter 104 can include a spring or memory element that imparts a spring-like deflection at the distal end 104a sufficient to establish good mechanical contact between the inner surface of the renal artery 12 and the electrodes 120 and transducers 130, 140 of the tissue assessment arrangement 115.

In accordance with various embodiments, the distal end 104a of the catheter 104 can include a multiplicity of tissue assessment arrangements 115. For example, the distal end 104a of the catheter 104 shown in FIG. 8 incorporates four tissue assessment arrangements 115 separated from each other by 90°. It is understood that more or fewer than four tissue assessment arrangements 115 can be incorporated with desired circumferential and/or axial separation. Incorporating a multiplicity of tissue assessment arrangements 115 provides for tissue monitoring for a circumferential region of the vessel 12, and can eliminate the need for repositioning the tissue assessment arrangements 115 during the ablation procedure. The configuration shown in FIG. 8 is particularly useful in embodiments that include an ablation arrangement at the distal end of the catheter 104, allowing for both tissue monitoring and ablation for a circumferential region of the vessel 12 without having to repositioning the catheter's distal end during the ablation procedure.

The catheter 104 of the system 100 shown in FIG. 7 includes an external system 200 which is communicatively coupled to the tissue assessment arrangement 115. The external system 200 includes a vibration source 202, which generates an acoustic source signal that impinges the target tissue, and a detector 204, which detect a return signal from the target tissue excited by the acoustic source signal. The detector 204 produces an output signal representative of the acoustic return signal, and communicates this signal to a processor 220.

The processor 220 is configured to implement algorithms for assessing one or more mechanical or electromechanical properties of the target tissue using the acoustic return signal. A user interface 230 is coupled to the processor 220 and generates various forms of output, including data, imaging, and other forms of information useful to a clinician. It is noted that the detector 204 may incorporate the processor 220 and be referred to herein as “the detector.” It is further noted that the processor 220 may incorporate the detector 204 and also be referred to herein as “the detector.” As such, the functions performed by the detector 204 and the processor 220 may be implemented by as a single component or multiple components.

The external system 200 further includes an ablation unit 210. The ablation unit 210 is electrically, fluidly, and/or optically coupled to the catheter 104 or a separate catheter arrangement for delivering and controlling ablation of target tissue. The ablation unit 210 can be implemented to deliver ablation therapy in accordance with various technologies. For example, and in accordance with various embodiments, the ablation unit 210 can include an RF generator for delivery of RF energy to the electrodes 120a and 120b. According to other embodiments, the ablation unit 210 can include a cryogen source and a pump for delivering a cryogen to one or more cryothermal elements provided at the distal end of the catheter 104 or a separate catheter arrangement. In further embodiments, the ablation unit 210 can include one or more ultrasound transducers (e.g., HIFU transducers) for delivering acoustic energy to the target tissue. The ultrasound transducers may be operated in a thermal ultrasound mode or a cavitating ultrasound mode for ablating the target tissue. In some configuration, one or more ultrasound transducers can be operated to provide multiple functionality, including delivery of ablation therapy, imaging or scanning of tissue, and interrogation of target tissue for assessing mechanical properties of the target tissue. In yet other embodiments, the ablation unit 210 can include one or more laser elements or a high-intensity flash lamp for delivering optical energy to the target tissue. The laser elements can be operated in a thermal or cavitating mode, for example.

These and other ablation technologies can be implemented to deliver ablation therapy to target tissue of the body in cooperation with a tissue assessment arrangement 115 in accordance with various embodiments. According to various embodiments, the system 100 shown in FIG. 7 can be implemented to automatically or semi-automatically monitor changes in soft tissue of the body subject to ablation and to control the ablation procedure, such as by adjusting the ablation energy or duration of energy delivery, terminating the ablation, adjusting delivery of cooling to tissue at the ablation site, and/or coordinating multiple ablation elements such as two or more RF electrodes, for example.

The vibration and/or monitoring mechanisms described herein can be combined with various types of ablation apparatuses using vibration or ultrasound, microwave, laser, or separate ablation mechanisms such as RF energy or cryothermal energy. A combination component, such as an RF electrode which also vibrates or monitors vibration, can be used to minimize the size of an intravascular device and position the ablation and the monitoring functions at the same location. Details of various ultrasound denervation therapy apparatuses and methods that can be implemented in accordance with embodiments of the disclosure are described in commonly owned U.S. Patent Publication No. ______, filed as U.S. patent application Ser. No. 13/086,116 on Apr. 13, 2011, which claims the benefit of U.S. Provisional Patent Application Ser. No. 61/324,164 filed Apr. 14, 2010 and entitled “Focused Ultrasound for Renal Denervation,” which are incorporated herein by reference. Embodiments that utilize a laser or a high intensity flash lamp are also contemplated. Details of these and other phototherapy denervation therapy apparatuses and methods that can be implemented in accordance with embodiments of the disclosure are described in commonly owned U.S. Patent Publication No. ______, filed as U.S. patent application Ser. No. 13/086,121 on Apr. 13, 2011, which claims the benefit of U.S. Provisional Patent Application Ser. No. 61/324,163 filed Apr. 14, 2010 and entitled “Phototherapy for Renal Denervation,” which are incorporated herein by reference.

Details of other denervation therapy apparatuses and methods that can be implemented in accordance with embodiments of the disclosure are described in commonly owned U.S. Patent Publication No. ______, filed as U.S. patent application Ser. No. 12/980,952 on Dec. 29, 2010, which claims the benefit of U.S. Provisional Patent Application Ser. No. 61/291,476 filed Dec. 31, 2009; U.S. Patent Publication No. ______, filed as U.S. patent application Ser. No. 12/980,972 on Dec. 29, 2010, which claims the benefit of U.S. Provisional Patent Application Ser. No. 61/291,480 filed Dec. 31, 2009; U.S. Patent Publication No. ______, filed as U.S. patent application Ser. No. 13/157,844 on Jun. 10, 2011, which claims the benefit of U.S. Provisional Patent Application Ser. No. 61/353,853 filed Jun. 11, 2010; and U.S. Patent Publication No. ______, filed as U.S. patent application Ser. No. 13/087,163 on Apr. 14, 2011, which claims the benefit of U.S. Provisional Patent Application Ser. No. 61/324,165 filed Apr. 14, 2010; each of which is incorporated herein by reference.

Refined imaging, such as elastography with a displayed image, can be used, or one or more simpler graphical displays or aggregate readings can be used to indicate that the appropriate ablation has occurred. These and other forms of output from the vibration and monitoring mechanism can be presented on the user interface 230. For example, the ablation monitoring can incorporate an indicator for the clinician, such as a constructed image, or line display, color-coded indicator, etc. Useful monitoring parameters can be presented via the user interface 230, including pulse waveform, time-lag, rise- or fall-slope, impulse response, damping, loss tangent, loss modulus, storage modulus, complex impedance, and ratios at different frequencies, for example. Transducers can perform both vibrating and sensing functions, or different transducers can be used for each function. Combined monitoring can be used to assess electrode contact with the artery wall 12 as well as ablation effect in the target tissue, as is described hereinbelow. Monitoring can be used to orient an asymmetric RF electrode for efficient ablation, for example, when positioning the tissue assessing arrangement 115 against the wall of the vessel 12.

Various embodiments of the disclosure are directed to apparatuses and methods for monitoring RF electrode contact with a vessel wall (e.g., renal artery) and changes in target tissue during ablation of target tissue (e.g., perivascular renal nerves). Conventional approaches typically monitor electrical impedance or electrode current as an indicator of electrode contact, but current flow through the blood and variations in tissue properties can make this approach less effective than desired. Conventional approaches typically use monitoring of the delivered electrical current or electrical impedance as a rough indicator of good electrode contact, but unknown electrical transmission to the blood, as well as variations in the local tissue, can make this approach less effective. Variable electrode contact with the artery wall can cause unpredictability in the ablation effect on the target tissue.

Embodiments of the disclosure are directed to apparatuses and methods that use mechanical force and displacement assessment of a vibrating transducer to monitor the progress of the ablation procedure so that effective ablation is obtained while minimizing excess injury to non-target tissue. According to various embodiments, an RF electrode arrangement on a catheter positioned within the renal artery can be vibrated while monitoring tissue displacement and the energy used. Comparison of displacement and force (or applied electrical current) values and waveforms provides for assessment of what is referred to as a kind of mechanical impedance. Embodiments that use mechanical force and displacement assessment of a vibrating transducer can incorporate various configurations of a tissue assessment arrangement 115 described herein.

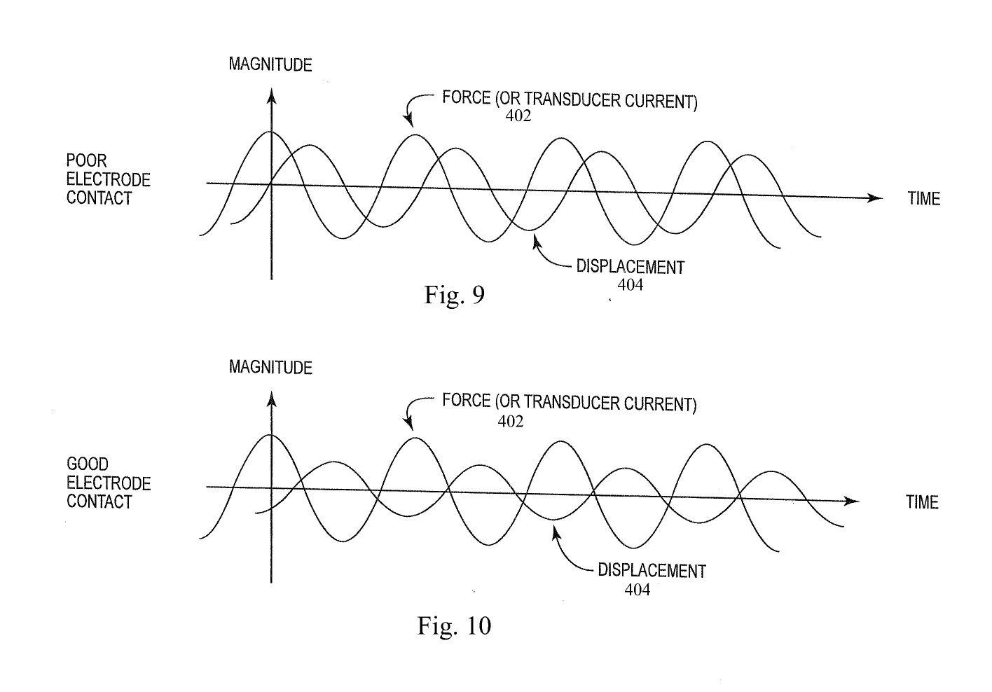

Reference is made to FIGS. 9 and 10, which graphically show displacement and force (or applied electrical current) relationships for poor and good electrode-to-tissue contact scenarios, respectively. FIGS. 9 and 10 graphically illustrate differences between force waveforms 402 and displacement waveforms 404 for a vibrated electrode 120. The apparatus for assessing mechanical force and displacement, such as that shown in FIG. 7, can be adapted to determine orientation of an electrode 120, and to assess the progress of ablation, as mechanical properties of the target tissue change during heating or freezing. As can be seen in FIGS. 9 and 10, the magnitude and phase lag of the force and displacement waveforms 402 and 404 vary as a result of differences in electrode-to-tissue contact integrity. Various parameters can be evaluated to assess electrode-to-tissue contact integrity, including pulse waveform, rise- or fall-slope. Other useful parameters include time-lag, impulse response, damping, loss tangent, loss modulus, storage modulus, complex impedance, ratios at different frequencies, among others.

Force can be measured directly or inferred from the current used to drive the vibration. Displacement can be measured by strain gauge voltage, accelerometer, variable capacitance or inductance in a movable structure coupled to the vibrating electrode, such as an electroactive material structure, or other means. Alternatively, displacement can be preset or fixed to a known pattern by the vibrating mechanism and only force (or electrical current) need be measured.

As can be seen in FIGS. 9 and 10, when the RF electrode 120 is in good contact with the artery wall 12, the electrode vibration displacement (represented by displacement waveform 404) will be decreased or the energy required to vibrate the electrode will be increased, for example. By assessing the electrode vibration displacement and applied force waveforms 404 and 402, the electrode-to-wall contact can be characterized. Low-frequency mechanical vibrations, sonic, or ultrasonic vibrations, for example, can be used with appropriate configuration of the vibrating electrode(s) 120 and vibration patterns. The vibration can continue during application of RF energy or other form of ablation energy, or intermittent assessment can be used, using vibration and RF energy alternately, for example. In various embodiments, and a discussed previously with reference to FIG. 7, mechanical electrode-to-tissue contact assessment can be used to automatically or semi-automatically control the ablation, such as by adjusting the ablation energy or energy delivery duration, terminating the ablation, adjusting cooling to tissue at the ablation site, or by coordinating multiple RF electrodes or other ablation elements, for example. Alternatively, the clinician can be informed of the mechanical assessment and make adjustments as needed.

Various embodiments of the disclosure are directed to apparatuses and methods for monitoring RF electrode contact with the artery wall, and effect on the target tissue, during ablation of perivascular renal nerves for treatment of hypertension. Embodiments of the disclosure are directed to apparatuses and methods that take advantage of mechanical and electrical impedance changes to assess RF electrode-to-tissue contact and monitor the progress of the ablation procedure so that effective ablation is obtained while minimizing excess injury to non-target tissue.

In various embodiments, an RF electrode on a catheter in the renal artery (see, e.g., FIGS. 7 and 8) is vibrated while monitoring the tissue displacement and energy used for vibration, and the voltage and current of RF energy are monitored as well. As was previously discussed, comparison of displacement and force (or applied electrical current) values and waveforms permits assessment of a kind of mechanical impedance; when the RF electrode is in good contact with the artery wall, the electrode vibration displacement will be decreased or the energy required to vibrate the electrode will be increased, for example.

Comparison of voltage and current of RF energy permits assessment of electrical impedance. Changes in tissue stiffness and electrical conductivity occur during ablation. By monitoring and comparing mechanical and electrical impedance, the electrode-to-wall contact can be characterized, and changes in the target tissue can be monitored during the ablation procedure. As previously discussed, low-frequency mechanical vibrations, sonic, or ultrasonic vibrations, can be used with appropriate configuration of the vibrating electrode(s) and vibration patterns. The vibration can continue during application of RF energy, or intermittent assessment can be used, using vibration and RF energy alternately.

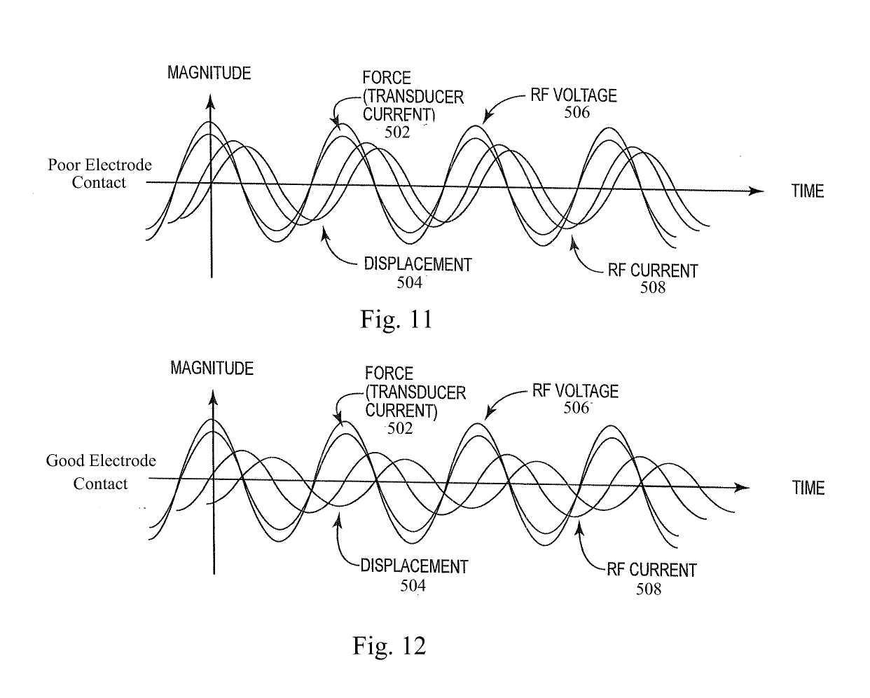

Reference is made to FIGS. 11 and 12 which graphically illustrate various waveforms being affected by good or poor RF electrode-to-tissue contact or by mechanical or conductivity changes in the target tissue. Differences in the waveforms, and differences in the mechanical and the electrical waveform changes, are used to determine contact and tissue changes. FIGS. 11 and 12 show mechanical and electrical waveforms associated with poor and good electrode-to-tissue contact, respectively. In particular, FIGS. 11 and 12 show two mechanical waveforms, a force (or transducer current) waveform 502 and a displacement waveform 504, and two electrical waveforms, an RF voltage waveform 506 and an RF current waveform 508.

As was previously discussed, comparison of displacement and force (or applied electrical current) values and waveforms 504, 502 permits assessment of a kind of mechanical impedance, while comparison of RF voltage and RF current values and waveforms 506, 508 permits assessment of electrical impedance. It can be seen in FIGS. 11 and 12 that when the RF electrode is in good contact with the artery wall, the electrode vibration displacement 504 and RF current 508 will be decreased or the energy required to vibrate the electrode will be increased, for example. It can further be seen in FIGS. 11 and 12 that when the RF electrode is in poor contact with the artery wall, the electrode vibration displacement 504 and RF current 508 will be increased or the energy required to vibrate the electrode will be decreased.

According to various embodiments, electromechanical impedance monitoring can be used to assess tissue changes during ablation, assess electrode contact, or to both assess electrode contact with a vessel wall and monitor ablation effect in target tissue, for example. In the context of various embodiments disclosed herein, electromechanical impedance may be characterized as a ratio of electrical impedance and mechanical impedance. The following equations for deriving electromechanical impedance in the context of various embodiments are provided for illustrative purposes, noting that the subscript “rf” (radiofrequency) is associated with electrical impedance, the subscript “mech” (mechanical) is associated with mechanical impedance (see, e.g., dynamic mechanical analysis or rheology), and the subscript EM is associated with electromechanical impedance:

Z_{rf}=V/I=Z*=Z_{rf-real}+R_{rf-imaginary}=resistance+reactance [1]

Z_{mech}=force/displacement=stress/strain=Loss Modulus+storage modulus [2]

Z_{EM}=Z_{rf}/Z_{mech} [3]

In the case of constant force in Equation [2] above, Z_{EM} can be derived from Z_{rf} * displacement or Z_{rf} * strain. In the case of constant force and voltage in Equation [2] above, Z_{EM} can be derived from strain/current. Electromechanical impedance can be used in several ways, including to detect and assess electrode-tissue contact, to estimate power delivered to tissue vs. blood (power for contact vs. no contact), and to increase sensitivity of tissue impedance changes associated with ablation (e.g., for lesion assessment), among others.

In the embodiments illustrated in FIGS. 9-12, positioning of the tissue assessment arrangement which includes one or more RF electrodes can be adjusted and the various waveforms and associated values evaluated. Establishing good electrode-two-tissue contact can be achieved by moving the RF electrode and evaluating the magnitude changes in the various waveforms. During electrode positioning, the peak magnitudes of each of the waveforms can be compared, and electrode positions associated with maximum and minimum peak magnitudes can be determined Having empirically determined electrode positions associated with reduced or minimum displacement and RF current waveform magnitudes, good or acceptable electrode contact locations can be determined. These determinations can be achieved automatically, such as by the processor of an external systems such as that shown in FIG. 7, or by clinician assessment.

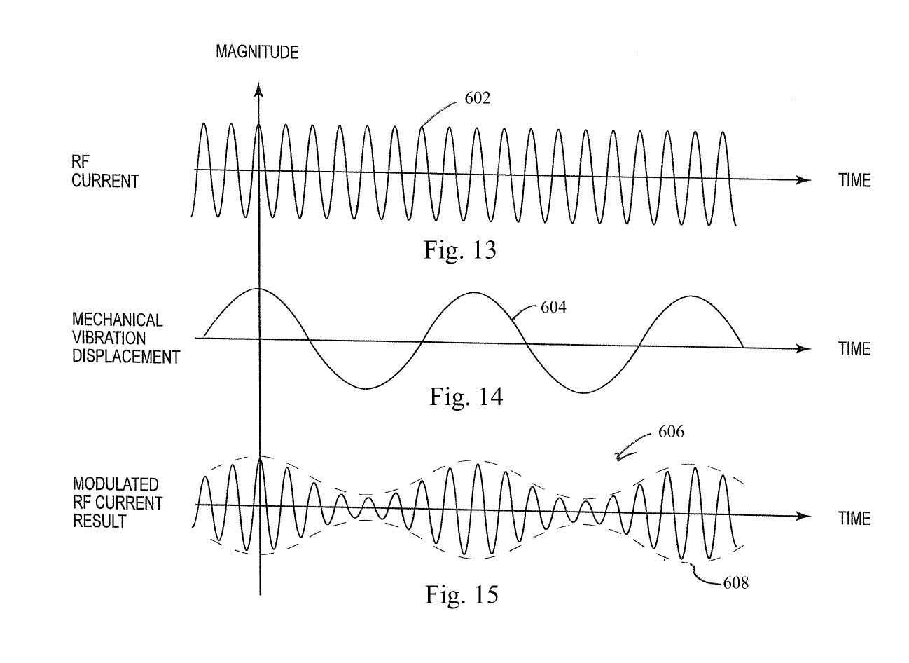

According to other embodiments, and with reference to FIGS. 13-15, changes in the mechanical vibration displacement waveform 604, corresponding to changes in mechanical properties of the target tissue during ablation, can be used to modulate an RF current waveform 602. According to such embodiments, high-frequency or RF electrical energy is used, such as the RF ablation energy or lower-voltage non-ablative energy, and mechanical changes from the mechanical vibration modulate the RF current or impedance waveform of the resulting RF energy. The resulting modulated RF current waveform 606 is shown in FIG. 15.

FIGS. 13-15 graphically illustrate the effect of mechanical vibration on modulating the electrical signals. These effects will be different if RF electrode-to-tissue contact is different, and if the tissue properties change as a result of ablation. Low-frequency mechanical vibrations, sonic, or ultrasonic vibrations, for example, can be used with appropriate configuration of the transducer(s), vibration intensities, and timings, as previously discussed. A modulator can be coupled to or incorporated in a vibration source (e.g., modulator 205 and vibration transducer 202 shown in FIG. 7). A waveform indicative of resulting mechanical vibration of the RF electrode, for example, can be used to modulate a waveform indicative of RF current supplied to the vibration source or an impedance waveform (e.g., electromechanical impedance) developed from RF supply current and voltage. In some embodiments, it may be desirable to include a demodulator coupled to or incorporated in the detector (e.g., demodulator 203 and detector 204 shown in FIG. 7). The detector can be configured to measure one or more parameters indicative of the effect of mechanical vibration on modulating the RF current or impedance waveform.

For example, the envelope 608 of the modulated RF current signal 606 can be evaluated for a number of positions of the electrode in contact with a vessel wall. The minimum and maximum magnitudes of the envelope 608, and the difference between these magnitudes, can be measured for each of the various electrode positions. Small differences between minimum and maximum magnitudes of the envelope 608 correspond to electrode positions having good electrode-to-tissue contact. Conversely, large differences between minimum and maximum magnitudes of the envelope 608 correspond to electrode positions having poor electrode-to-tissue contact.

In various embodiments, electromechanical impedance monitoring can be used to automatically or semi-automatically control the ablation procedure, such as by adjusting the ablation energy or duration of energy delivery, terminating the ablation, adjusting delivery of cooling to tissue at the ablation site, and/or coordinating multiple RF electrodes, for example. Alternatively, the clinician can be informed of the mechanical assessment and make adjustments as needed. Pulse waveform, time-lag, or rise- or fall-slope or other parameters can be used to facilitate the monitoring, and relative differences between mechanical and electrical impedance changes can be used to assess property changes in the target tissue during ablation. Additional useful parameters include impulse response, damping, loss tangent, loss modulus, storage modulus, complex impedance, and ratios of any of these parameters at different frequencies, for example.

In some embodiments, electromechanical impedance monitoring can be used solely to assess electrode contact, or solely to assess tissue changes during ablation. Alternatively, electromechanical monitoring can be used to both assess electrode contact with the artery wall and monitor ablation effect in the target tissue. Monitoring functions can be used continuously during RF energy application, or can be used intermittently, or can alternate with RF energy application. Electromechanical monitoring can also be used to orient an asymmetric RF electrode, such as one with a conductive surface (towards the vessel wall) and an insulated surface (towards the blood) for efficient ablation.

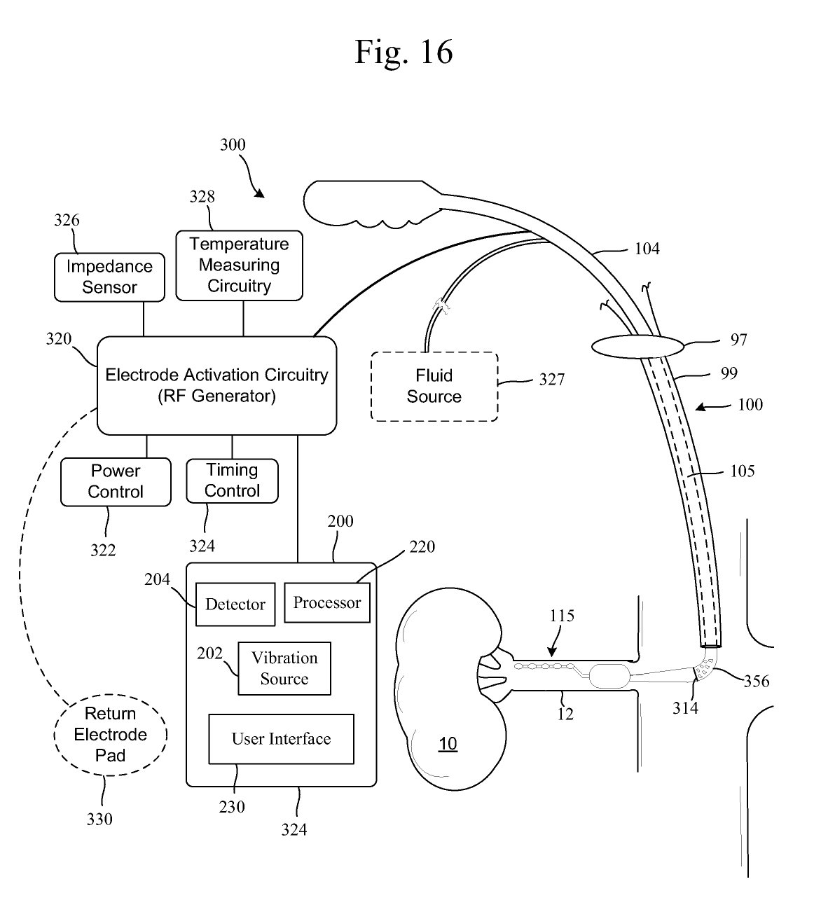

FIG. 16 shows a representative renal ablation apparatus 300 in accordance with various embodiments of the disclosure. Although the apparatus 300 is configured for RF ablation, it is understood that the apparatus 300 can be configured to delivery other forms of ablative energy, such as ultrasound, optical, and cryothermal energy, for example. The apparatus 300 illustrated in FIG. 16 includes external electrode activation circuitry 320 which comprises power control circuitry 322 and timing control circuitry 324. The external electrode activation circuitry 320, which includes an RF generator, is coupled to temperature measuring circuitry 328 and may be coupled to an optional impedance sensor 326. The catheter 104 includes a shaft that incorporates a lumen arrangement 105 which can be configured for receiving a variety of components, such as conductors, pharmacological agents, actuator elements, obturators, sensors, or other components as needed or desired. The catheter 104 can be delivered to the renal artery 12 using a guide sheath or guiding catheter 99 via a percutaneous access location 97. The catheter 104 may include a hinge mechanism 356 to aid in navigating the catheter around the nearly 90° turn from the aorta and into the renal artery 12.

The RF generator of the external electrode activation circuitry 320 may include a return pad electrode 330 that is configured to comfortably engage the patient's back or other portion of the body near the kidneys. In this configuration (unipolar), a single RF electrode 120 may be situated at the distal end of the catheter 104. In a bipolar configuration, at least two RF electrodes 120 are situated at the distal end of the catheter 104, in which case the return electrode pad 330 is not needed. Radiofrequency energy produced by the RF generator is coupled to a tissue assessment arrangement 115 at the distal end of the catheter 104 by a conductor arrangement disposed in the lumen of the catheter's shaft. The radiofrequency energy flows through the electrode(s) 120 in accordance with a predetermined activation sequence (e.g., sequential or concurrent) to ablate perivascular renal nerves adjacent the renal artery 12. In general, when renal artery tissue temperatures rise above about 113° F. (50° C.), protein is permanently damaged (including those of renal nerve fibers). If heated over about 65° C., collagen denatures and tissue shrinks. If heated over about 65° C. and up to 100° C., cell walls break and oil separates from water. Above about 100° C., tissue desiccates.

According to some embodiments, the processor 220 of the external system 200 is configured to perform mechanical and/or electromechanical assessment of target tissue in accordance with the various techniques described herein. The external system 200 in cooperation with the transducers 130, 140 and RF electrode(s) 120 of the tissue assessment arrangement 115 can be used to determine optimal or adequate electrode positions prior to initiating the ablation procedure. The tissue assessment arrangement 115 may further be used in cooperation with the electrode activation circuitry 320 to automatically or semi-automatically control the ablation procedure.

The electrode activation circuitry 320 is configured to control activation and deactivation of the electrode(s) 120 in accordance with a predetermined energy delivery protocol and in response to signals received from the processor 220, the temperature measuring circuitry 328, and/or the impedance sensor 336. The electrode activation circuitry 320 controls radiofrequency energy delivered to the electrodes 120 so as to maintain the current densities at a level sufficient to cause heating of the target tissue to at least a temperature of 55° C., for example. A cooling fluid dispensed by a fluid source 327 may be delivered to the distal end of the catheter 104 to provide cooling at the electrode-tissue interface. The cooling source 327 may be controlled automatically by the electrode activation circuitry 320 or the processor 220.

In some embodiments, temperature sensors are situated at the distal end of the catheter 104 and provide for continuous monitoring of renal artery tissue temperatures, and RF generator power is automatically adjusted so that the target temperatures are achieved and maintained. An impedance sensor arrangement 326 may be used to measure and monitor electrical impedance during RF denervation therapy, and the power and timing of the RF generator 320 may be moderated based on the impedance measurements or a combination of impedance, temperature measurements, and tissue assessment output signals communicated from the detector 204 to the processor 220.

Marker bands 314 can be placed on one or multiple parts at the distal end of the catheter 104 to enable visualization during the procedure. Other portions of the catheter 104, such as one or more portions of the shaft 104 (e.g., at the hinge mechanism 356), may include a marker band 314. The marker bands 314 may be solid or split bands of platinum or other radiopaque metal, for example, to aid in catheter navigation and electrode positioning. A braid and/or electrodes or sensors of the catheter 104, according to some embodiments, can be radiopaque.

Various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vessels (e.g., cardiac and urinary vasculature and vessels), and other tissues of the body, including various organs. For example, various embodiments may be configured to treat benign prostatic hyperplasia (BPH) or to diagnose and/or treat a tumor using an appropriate medical device advanced to the treatment site through an appropriate body pathway. Embodiments of the disclosure can be implemented for use in a variety of ablation procedures involving the heart or cardiac vessels, such as for cardiac arrhythmia therapy, for example. Embodiments of the disclosure can be implemented to position other ablation devices in intimate contact with tissue, including positioning intravascular therapy devices, urological devices, devices in a heart chamber, devices in the gastrointestinal tract, among others.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a catheter apparatus having a length sufficient to access target tissue of the body relative to a percutaneous access location;
a transducer arrangement supported at least in part by the catheter apparatus, the transducer arrangement comprising:
a vibrating transducer configured to cause the target tissue to vibrate; and
a sensing transducer configured to sense vibration of the target tissue caused by the vibrating transducer; and
a detector communicatively coupled to transducer arrangement, the detector configured to measure changes in elasticity of the target tissue and produce an output signal indicative of the measured changes in target tissue elasticity.

2. The apparatus according to claim 1, wherein the sensing transducer comprises a plurality of sensing transducers or a transducer array.

3. The apparatus according to claim 1, wherein the detector is configured to measure changes in tissue elasticity due to application of ablation energy to the target tissue.

4. The apparatus according to claim 1, wherein each of the vibrating transducer and the sensing transducer comprises an acoustic transducer.

5. The apparatus according to claim 1, wherein at least one of the vibrating and sensing transducers is configured for extravascular or patient-external deployment.

6. The apparatus according to claim 1, wherein each of the vibrating and sensing transducers is configured for intravascular deployment.

7. The apparatus according to claim 1, wherein the detector is configured to monitor one or more parameters of an acoustic signal produced by the sensing transducer.

8. The apparatus according to claim 1, wherein the detector is configured to monitor one or more parameters of an acoustic signal produced by the sensing transducer, the one of more parameters comprising one or more of a pulse waveform, a time-lag, a rise- or fall-slope, an impulse response, a damping, a loss tangent, a loss modulus, a storage modulus, a complex impedance, and ratios at different frequencies.

9. The apparatus according to claim 1, further comprising an ablation arrangement and a processor communicatively coupled to the detector, the processor configured to monitor changes in target tissue elasticity during an ablation procedure using the output signal produced by the detector.

10. The apparatus according to claim 1, wherein the target tissue comprises tissue of a vessel, tissue of an organ, tissue of a tumor, diseased tissue.

11. The apparatus according to claim 1, wherein:
the vibrating transducer is configured to direct high-frequency acoustic energy to the target tissue; and
the sensing transducer is configured to sense a low-frequency return signal or image which includes signal content corresponding to vibration of the target tissue caused by the vibrating transducer.

12. The apparatus according to claim 1, wherein:
the vibrating transducer is configured to direct low-frequency acoustic energy to the target tissue; and
the sensing transducer is configured to sense a high-frequency return signal or image which includes signal content corresponding to vibration of the target tissue caused by the vibrating transducer.

13. An apparatus, comprising:
a catheter apparatus having a lumen and a length sufficient to access a patient's renal artery relative to a percutaneous access location;
an ablation arrangement configured to ablate perivascular renal nerve tissue;
a transducer arrangement supported at least in part by the catheter apparatus, the transducer arrangement comprising:
a vibrating transducer configured to cause the perivascular renal nerve tissue to vibrate; and
a sensing transducer configured to sense vibration of the perivascular renal nerve tissue caused by the vibrating transducer; and
a detector communicatively coupled to transducer arrangement, the detector configured to measure changes in elasticity of the perivascular renal nerve tissue due to ablation and produce an output signal indicative of the measured changes in perivascular renal nerve tissue elasticity.

14. The apparatus according to claim 13, wherein the ablation arrangement comprising one or a combination of one or more RF electrodes, one or more cryothermal elements, one or more ultrasound elements, and one or more phototherapy elements.

15. The apparatus according to claim 13, further comprising a processor communicatively coupled to the detector and the ablation arrangement, the processor configured to monitor changes in perivascular renal nerve tissue elasticity due to ablation using the output signal produced by the detector, and adjust a parameter of one or both of the ablation arrangement and the transducer arrangement during ablation using the output signal produced by the detector.

16. An apparatus, comprising:
a catheter apparatus having a length sufficient to access target tissue of the body relative to a percutaneous access location;
an RF electrode supported by the catheter apparatus and configured to contact the target tissue;
a transducer arrangement supported at least in part by the catheter apparatus, the transducer arrangement comprising:
a vibrating transducer configured to emit acoustic energy that causes the RF electrode to vibrate; and
a sensing transducer configured to sense an acoustic wave indicative of displacement of the RF electrode caused by the emitted acoustic energy; and
a detector communicatively coupled to the transducer arrangement, the detector configured to generate an output indicative of a force applied to the RF electrode by the emitted acoustic energy and displacement of the RF electrode.

17. The apparatus of claim 16, wherein:
the output comprises one of values and waveforms indicative of the force applied to the RF electrode by the emitted acoustic energy and the displacement of the RF electrode; and
the detector is configured to generate additional output indicative of electrode-to-tissue contact integrity based on a comparison of the values or waveforms indicative of the force applied to the RF electrode by the emitted acoustic energy and the displacement of the RF electrode.

18. The apparatus of claim 16, wherein:
the output comprises one of values and waveforms indicative of (a) the force applied to the RF electrode by the emitted acoustic energy, (b) the displacement of the RF electrode, (c) RF voltage supplied to the RF electrode, and (d) RF current supplied to the RF electrode; and
the detector is configured to generate additional output indicative of electrode-to-tissue contact integrity based on a comparison of the values or waveforms indicative of (a) the force applied to the RF electrode by the emitted acoustic energy and (b) the displacement of the RF electrode, and a comparison of the values or waveforms indicative of (c) the RF voltage supplied to the RF electrode and (d) the RF current supplied to the RF electrode.

19. The apparatus of claim 16, further comprising:
a vibration source coupled to the vibrating transducer; and
a modulator coupled to or incorporated in the vibration source, the modulator configured to modulate a waveform indicative of RF current supplied to the vibration source or an impedance waveform developed from RF supply current and voltage;
wherein the detector is configured to measure one or more parameters indicative of an effect of RF electrode vibration on modulating the RF current or impedance waveform.

20. The apparatus according to claim 16, wherein:
the vibrating transducer is configured to emit high-frequency acoustic energy to the target tissue; and
the sensing transducer is configured to sense a low-frequency return signal or image which includes signal content indicative of displacement of the RF electrode caused by the emitted acoustic energy.

21. The apparatus according to claim 16, wherein:
the vibrating transducer is configured to emit low-frequency acoustic energy to the target tissue; and
the sensing transducer is configured to sense a high-frequency return signal or image which includes signal content indicative of displacement of the RF electrode caused by the emitted acoustic energy.

22. A method, comprising:
causing target tissue of the body to vibrate;
sensing vibration of the target tissue;
measuring changes in elasticity of the target tissue based on the sensed vibration; and
producing an output indicative of the measured changes in target tissue elasticity.

23. The method of claim 22, further comprising:
ablating the target tissue;
measuring changes in elasticity of the target tissue due to ablation; and
producing an output indicative of the measured changes in target tissue elasticity due to ablation.

24. A method, comprising:
causing an electrode in contact with target tissue of the body to vibrate;
sensing vibration of the electrode;
measuring a force applied to the electrode caused by electrode vibration;
measuring displacement of the electrode resulting from electrode vibration; and
producing an output indicative of the force applied to the electrode and the displacement of the electrode.
